(19) United States
(12) Patent Application Publication
(10) Pub. No.: US 2014/0087448 A1
(43) Pub. Date: Mar. 27, 2014

(54) PROCESS FOR OBTAINING A BROWN ALGAE LYOPHILISATE, AND COSMETIC PREPARATIONS THEREOF

(71) Applicant: BIOTECH MARINE, Pontrieux (FR)

(72) Inventor: Nicole MEKIDECHE, Ploubazlanec (FR)

(73) Assignee: BIOTECH MARINE, Pontrieux (FR)

(21) Appl. No.: 14/092,180

(22) Filed: Nov. 27, 2013

Related U.S. Application Data
(62) Division of application No. 11/813,861, filed on Jul. 12, 2007, now abandoned.

(30) Foreign Application Priority Data
Jan. 14, 2005 (FR) 0500389

(51) Int. Cl. A61K 8/97

(52) U.S. Cl. CPC A61K 8/975
USPC 435/257.1

(57) ABSTRACT

A process for obtaining a freeze-dried product of gametophytes cells of brown algae, and a cosmetic preparation for tropical use including the freeze-dried product of gametophytes cells of brown algae obtained.

This invention relates to a freeze-dried product of cells of brown algae, the process for obtaining thereof, a cosmetical composition containing them, a food complement containing them and uses thereof.

### FIELD OF INVENTION

The class of brown algae, also called Fucophycae or Phaeophyceae, belongs to the Division Chromophyta or “colored algae”, frequently called chrysophyta. This Division groups algae the cells of which contain “accessory” carotenoid pigments, such as fucoxanthin, in addition to chlorophyll pigments: chlorophyll a and c.

The class of brown algae comprises the orders of: Ascoseirales, Chordariales, Cutleriales, Desmarestiales, Dictyosiphonales, Dictyotales, Durvilleales, Ectocarpales, Fucales, Laminariales, Notheiales, Scytosiphonales, Sphacelariales, Sporochnales, Syringodermatales, Tilopteridales.

All photosynthetic organisms use the pigments to harvest the light energy, usually a form of chlorophyll. The standard chlorophyll is the chlorophyll a, and it is essential for the transfer of the captured light energy to the molecules which will make use of this energy. Most of the chlorophyll organisms have other pigments to absorb more light but the harvested energy must always be transferred to a molecule of chlorophyll a.

The brown algae make use of several such “accessory” pigments such as the chlorophyll c and the carotenoids. The Phaeophyceae exhibit great amounts of carotenoids in their plastids and these are the brown and yellow pigments which impart them this characteristic brown colour. The most important carotenoid pigment of the brown algae is fucoxanthin, the name of which originates from Fucus. The fucoxanthin absorbs wavelengths of 500 to 580 nm.

Carotenoid pigments have an aliphatic or alicyclic structure. They are soluble in fats, what furthers their direct integration into certain membranes. Therefore, their solubility in water can only arise when they are bound to other molecules. This is also the reason that their metabolism is most frequently in direct relation to the metabolism of lipids.

Carotenoids are also called accessory pigments, since they are bound to transfer the energy harvested by chlorophyll a. These pigments are generally known through the caroten which gave its name to this family of pigments.

The only source of carotenoids, particularly for animals and humans, is their food. The carotenoids have an antioxydant function in the human plasma, especially for the circulating lipids. This function is expressed also at the cell membrane level where they are incorporated. The carotenoids have a direct part in the coloration of the skin (yellow-orange) imitating the natural tan.

### PRIOR ART

Caroten is used as food complement to be taken a few days before and during the exposition to sunlight in order to accelerate the tan. The beta-caroten is the precursor of the vitamin A. The intake of beta-caroten delays the outbreak of solar erythema. Vitamin A increases the amount of melanin produced under the action of a blue light. Further, the vitamin A has an anti-oxydative protective role. The vitamins A and E avoid the oxydative denaturation of the melanin under the action of UV-light.

In the known food complements, caroten is associated to the vitamin C, the vitamin E or to the flavonoids.

The drawback to the use of caroten for colouring the skin is that the resulting colour is much more orange-coloured than the natural tan. In addition, it is rapidly degraded to the vitamin A.

Astaxanthin was shown to be a powerful anti-oxydant. The U.S. Pat. No. 6,433,025 describes the oral use thereof for preventing and delaying sunburns.

Canthaxanthin, a further carotenoid pigment, was used as food complement for colouring the skin and for protecting thereof against UV rays (GB 1 323 800). Nevertheless, this use of canthaxanthin is prohibited in France because of the danger of blindness while it is currently marketed in the U.S. (Canthorex, DELTA®Laboratories).

Skin cancer or melanoma will be, in France, in the next years, the most frequent and is already in the U.S. with 800 000 cases/year. Most frequently, it is the consequence of an excessive proliferation of skin cells resulting from DNA mutations induced by ultraviolet light (UV).

People become more and more aware of this danger and are ready to protect themselves against the sun. Nevertheless, a tanned skin is still an advantageous and desired feature satisfying present esthetical criteria.

Therefore, the manufacturers of cosmetics and pharmaceutical preparations are currently investigating active substances for pigmenting the skin and for protecting the skin against the deleterious effects of the UV.

### SUMMARY OF THE INVENTION

The Applicant has surprisingly and unexpectedly discovered that a freeze-dried product of cells of brown algae, in particular of gametophytes, enriched in fucoxanthin, allows to achieve this combination of desired effects: it pigments the epidermis in an entirely harmless way by stimulating the melanogenesis, even without UV irradiation, while protecting it by an anti-radical activity and stimulating its regeneration.

In fact, brown algae have a complex digenetic cycle. For Laminariales (see FIG. 1), the alternation between a diploid sporophyte developed in a macroscopic thalle and the microscopic male gametophytes and female haploids, can be observed. In this case, gametophytes are only a set of cells resulting from different mitoses of the spore.

The gametophyte is transient with time and is only a stage of production of gametes. As it is not developed into a true thalle, it is weakly photosynthetic and contains a maximum amount of protecting pigments: fucoxanthin, compared to the true photosynthesis pigments which are chlorophylls.

Thus, the Applicant has selected this stage of gametophyte in order to obtain a freeze-dried product of cells of brown algae with an extrement enrichment in fucoxanthin, easily and at low cost, compared to a classical extraction of fucoxanthin, from a biomass available in unlimited amounts and throughout the whole year.

### DETAILED DESCRIPTION OF THE INVENTION

Thus, the first subject-matter of the present invention is a freeze-dried product of cells of brown algae enriched in fucoxanthin, in particular comprising at least 1% of fucoxanthin. Said cells of brown algae are preferably spores or gametophytes, in a particularly preferred manner, gametophytes.

The second subject-matter of the present invention is a process for obtaining a freeze-dried product of gametophytes of cells of brown algae comprising the following steps:

harvesting mature sporophytes

emission of spores in vitro

germination of spores in vitro

harvesting gametophyte cells

freeze-drying of the obtained gametophyte cells.

“Mature sporophyte” means a sporophyte comprising spores in special receptacles, for example sores.

The third subject-matter of the present invention is a cosmetic preparation for topical use comprising a freeze-dried product according to the invention as active ingredient, preferably in an amount ranging from of 0.2 to 5% in weight, more preferably, of 1 to 2% in weight.

The fourth subject-matter of the present invention is a food complement comprising a freeze-dried product according to the invention as active ingredient.

The cosmetic preparation according to the invention can be used for pigmenting the skin, for preparing the skin to be exposed to UV rays, for protecting the skin against oxydative stress induced by UV rays and/or for protecting the skin against cellular aging.

The food complement according to the invention can be used for preparing the skin to be exposed to UV rays.

### DESCRIPTION OF FIGURES

FIG. 1: reproduction cycle of Laminariales

FIG. 2: sections of epidermis treated/untreated stained with HES (hematoxylin/eosine/safran).

### EXAMPLE 1

### Obtention of Gametophytes of Laminarial Brown Algae

Gametophytes cells were obtained for the following species: Laminaria saccharina, Laminaria hyperborea, Alaria esculenta, Undaria pinnatifida.

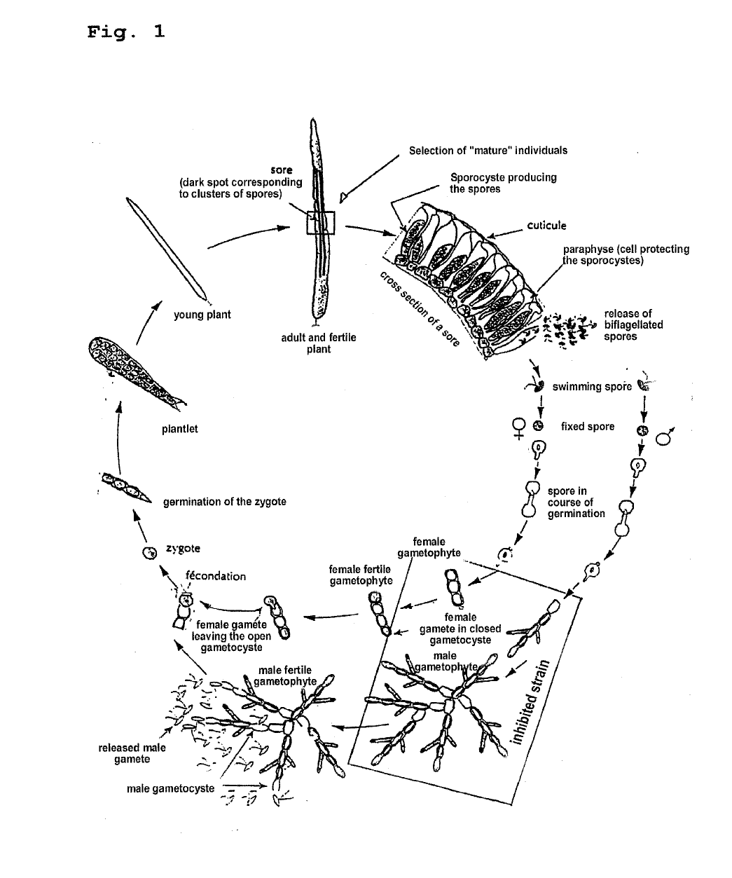

Thalli of fertile mature sporophytes were harvested, exhibiting sores (see FIG. 1) at JO on French coasts in Brittany, between October and February.

They are washed with filtered sea water and cut into pieces of 5 to 10 cm² which are successively dipped into filtered sea water, bleach 0.2% for 20 to 30 sec, in two tanks with filtered sea water.

Then, the pieces of thalle were dried on absorbent paper, distributed by 30 on a sheet of absorbent paper and the sheets of absorbent paper were rolled.

The sheets of rolled absorbent paper containing the pieces of thalli are incubated for 12 h at 15° C. Then they are placed into filtered sea water and afterwards, spores are emitted.

The spores are incubated in a defined culture medium containing 0.1% of Provasoli solution (see Table 1) in a flask in the presence of light (1800-2000 lux, 24 h/24), with slight shaking.

The temperature is increased by a half degree by day up to 22° C. The medium was renewed fortnightly.

The spores germinate after 6 to 20 days of culture and the gametophytes are harvested after 10-12 days following the germination (see Table 2).

TABLE 1
Composition of the Provasoli solution for 1 L

| | |
|---|---|
| EDTA | 3 g |
| Fe(Cl) | 0.08 g |
| Mn(Cl) | 0.12 g |
| Zn(Cl) | 0.015 g |
| Co(Cl) | 0.003 g |
| Ca(SO₄) | 0.0012 g |
| B(H₃BO₃) | 0.6 g |
| Mo(Na₂MoO₄) | 0.05 g |

TABLE 2

| | Germination period | Obtention of |
|---|---|---|
| Species | in days | gametophytes at |
| Laminaria | 9 | J19-J21 |
| saccharina | | |
| Laminaria | 11-20 | J21-J32 |
| hyperborea | | |
| Alaria esculenta | 6-7 | J16-J19 |
| Undaria | 7 | J17-J19 |
| pinnatifida | | |

### EXAMPLE 2

### Obtaining of a Freeze-Dried Product of Gametophytes Cells of Brown Algae

Inhibition of the gametophyte with a kanamycine-sulfate- and germanium dioxide-free medium for preventing the germination of the plantlet.

The culture is filtered on a sieve, the cells are rinsed with sea water and freeze-dried in a plate-freeze-drier.

In order to titer the fucoxanthin by HPLC, an ethanol extraction is performed at a rate of 0.05% of freeze-dried product for 10 ml of 70% ethanol, stirring for 6 h in darkness, then filtration.

HPLC analysis is performed on a Absorbsphere (Alltech) column with a solvent/gradient:

A=Ammonium acetate/methanol (20:80)

### B=Acetonitril 90%

C=Ethyl acetate

### EXAMPLE 3

### Preparation of the BB Product

A freeze-dried product of gametophytes of the seaweed Undaria pinnatifida (Laminariaciae) is prepared according to Example 2.

In order to use this freeze-dried product in a suitable way on skin culture, the freeze-dried product is put back into the culture medium of gametophytes. This mixture is ultrasonicated, then filtered. The result of this filtered product is called <<BB>>.

### EXAMPLE 4

### Evaluation of Mucous Tolerance of a Freeze-Dried Product of Gametophytes Cells of Brown Algae in Cytotoxicity Studies on the Reconstructed Cornea Skinethic®

### 1.—Experimental Protocol

Keratinocytes of the TR 146 line, spontaneously transformed, amplified, by cell culture in a defined modified MCDB 153 medium were used.

When cultured on the interface air/liquid in a defined medium, these human keratinocytes form a multilayered epithelium without the cornea layer similar to the human cornea.

The product to be investigated, in this case the BE product according to Example 3, is applied at a rate of 30 μL onto the surface of eight equivalent cultures using a micro-pipette. These cultures are subsequently incubated at 37° C., 5% of CO₂, for 10 minutes, 1 hour, 3 hours and 24 hours, at a rate of two cultures by incubation period.

Negative (buffered saline solution) and positive (SDS 0.5% and 1%) controls are prepared sterile and applied in the same way on two cultures, respectively.

These cultures are subsequently incubated at 37° C., 5% of CO₂, for 1 hour and 24 hours, at a rate of two cultures by incubation period.

### 2.—Assessment of the Cellular Viability

The cellular viability is further measured qualitatively after labelling with a vital dye. The MTT system measures the mitochondrial dehydrogenase activity of living cells. The key component is the 3-[4,5-dimethylthiazol-2-yl]-2,5-diphenyl tetrazolium bromide or MTT.

The salt solutions buffered with MTT, in the absence of phenol red, are yellow coloured. The mitochondrial deshydrogenases of living cells cut the tetrazolium cycle, thus inducing formation of purple formazan crystals, insoluble in aqueous solutions.

The crystals formed by the viable cells are trapped in the polycarbonate filters which serve as support to epithelial cultures. The cultures turn to a uniform intense blue/purple colour when they are viable, but remain white/yellow in the case of cell mortality.

The results are compared to negative and positive control substances.

0.15 ml of culture medium containing 10% vol/vol of MTT solution are pipetted under each filter/culture support. After an incubation of 30 min at room temperature, the colour of the different cultures is observed.

Negative control cultures should show an intense blue/purple colour, proof of the viability of mucosa after being in contact for 24 hours.

Positive control cultures should show a white colour, proof of the cellular mortality as from the 1^{st} hour of contact.

### 3.—Results

TABLE 3

| | Colour of both cultures | | | | |
|---|---|---|---|---|---|
| PRODUCT | 10 min | 1 h | 3 h | 24 h | TOXICITY |
| BB | blue | blue | blue | white | NI |
| Negative control | blue | blue | blue | blue | NI |
| SDS 0.5% | blue | white | white | / | I |
| SDS 1% | white | white | / | / | VI |

NI = not irritant
I = irritant
VI = very irritant

### 4.—Conclusion

Under the applied experimental conditions, the BB product according to Example 3, investigated pure, is not irritant to cells forming the model SKINETHIC® of in vitro reconstructed mucous membrane.

### EXAMPLE 5

### Evaluation of the Cutaneous Tolerance of a Freeze-Dried Product of Gametophytes Cells of Brown Algae, by Study of the Cytotoxicity On Reconstructed Epidermal Skin Skinethic®

### 1—Experimental Protocol

Keratinocytes of human origin are plated out on polycarbonate filters of 0.63 cm² in a defined and supplemented medium (modified MCDB 153). The cells are cultured for 10 days on the air/liquid interface, the culture medium being renewed every two days.

The thus formed epidermis were used for carrying out the study from the 14^{th} day of culture.

The test was performed in triplicate after incubation for 24 hours of the product, at a rate of 2 μl by epidermis.

The control epidermis did not receive any product.

Epidermis fixed in a 10% formaldehyde solution were embedded into paraffin blocs. Vertical sections of 4 microns were stained with HES (hematoxylin/eosine/safran) and photographs were taken under light microscope.

If the product is not toxic, the cultures should show basal, spineous, granular cell layers and intact orthokeratosic cornea, and the skin stratification should be regular and normal. The basal layer cells should be polarized vertically. Numerous keratohyalin grains should be visible (in violet) in the stratum granulosum just under the stratum corneum.

### 2—Results

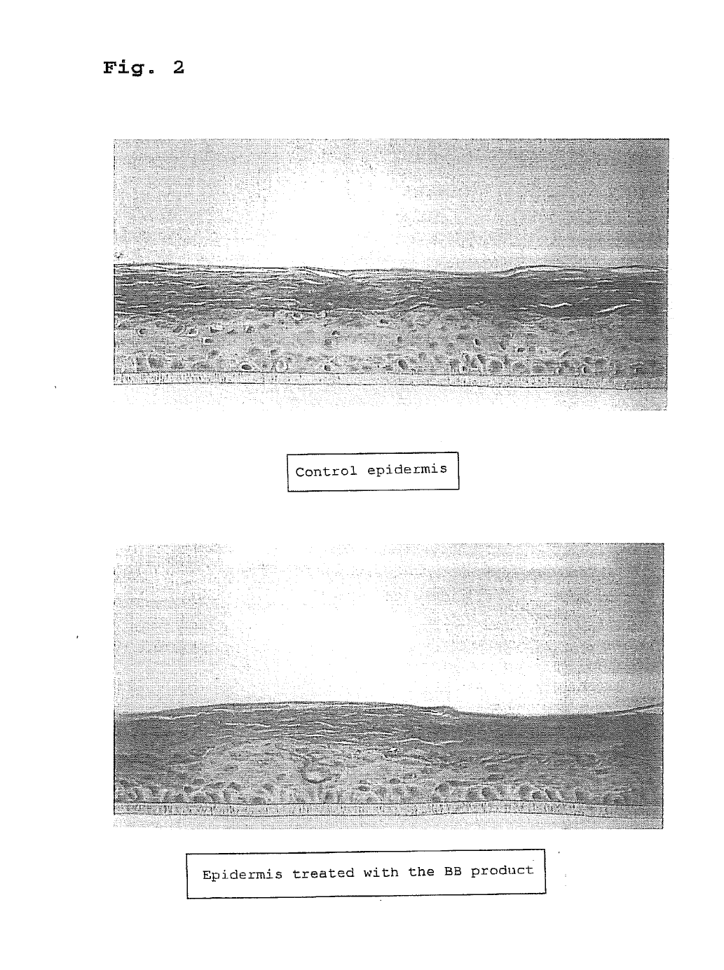

cf. FIG. 2

### 3.—Conclusion

Under the applied experimental conditions, the BB product according to Example 3, investigated pure, has been shown not to be cytotoxic on reconstructed skins SKINETHIC®.

### EXAMPLE 6

### Pigmentation Tests

### 1.—Materials and Methods

The BB product is diluted in ethanol then incorporated into a cosmetic formulation.

The tested products are:

a—BB product incorporated in formulation at 0.05%

b—BB product incorporated in formulation at 0.15%

c—BB product incorporated in formulation at 0.30%

They are conserved at 4° C. until use thereof.

### 2.—Biochemical Assessment

At the end of the incubation, the melanin contained in cell lysates was quantified by a spectrophotometrical method. A range of melanin was carried out in parallel with the assay.

The results are expressed in μg of melanin per ml of lysate of the cell carpets (average +/− standard deviation SD).

The statistical significance of the observed differences between the conditions [symbol] treated [symbol] and [symbol] controls [symbol] was assessed by variance analysis to a factor (One Way ANOVA) followed by a test of Bonferroni t-test (*:p<0.05;**:p<0.01).

TABLES 4, 5 and 6
biochemical quantification of the melanic content in reconstructed
melanized human epidermis treated/not treated:

| | | α- | ETOH BB | | | |
|---|---|---|---|---|---|---|
| | Medium | MSH | | | | 0.15 without |
| | alone | 100 nM | 0.05 | 0.15 | 0.3 | re-application |
| WITHOUT | | | | | | |
| UV | | | | | | |
| Melanin | 47.3 | 70.3 | 56.6 | 59.9 | 69.1 | 70.3 |
| (μg/ml) | 67.3 | 86.9 | 58.7 | 60.5 | 77.2 | 69.0 |
| | 75.8 | 85 | 56.1 | 66.1 | 56 | 58.5 |
| Average | 57.3 | 80.7 | 57.1 | 62.2* | 73.1** | 69.6* |
| S.D. | 14.1 | 9.1 | 1.3 | 3.4 | 5.7 | 0.9 |
| % medium | 100 | 141 | 100 | 108 | 128 | 122 |
| alone | | | | | | |
| % ETOH BB | | | 100 | 108.8 | 128 | 121.9 |
| 0.05 | | | | | | |
| +UVA | | | | | | |
| Melanin | 57.4 | 95.6 | 69.7 | 63.5 | 64.7 | 61.2 |
| (μg/ml) | 73.9 | 84.7 | 66.3 | 64.1 | 66.3 | 66.3 |
| | 73.4 | 107.9 | 57.3 | 65.7 | | |
| Average | 68.2 | 96.1 | 64.5 | 64.4 | 65.5 | 62.6 |
| S.D. | 9.4 | 11.6 | 6.4 | 1.2 | 1.2 | 3.3 |
| % medium | 100 | 141 | 94 | 94 | 96 | 92 |
| alone | | | | | | |
| +UVB | | | | | | |
| Melanin | 82.6 | 99.5 | 60.7 | 64.6 | 40.1 | 44.8 |
| (μg/ml) | 91.4 | 115.5 | 65.8 | 66.1 | 30.9 | 65.3 |
| | 75.3 | 104.6 | 67.1 | 70.5 | 38.7 | 64.5 |
| Average | 83.1 | 106.5 | 64.5 | 67.1 | 36.6 | 58.2 |
| S.D. | 8 | 8.2 | 3.4 | 3.1 | 5 | 11.6 |
| % medium | 100 | 128 | 78 | 81 | 44 | 70 |
| alone | | | | | | |

### 3.—Histological Assessment

The epidermis skins ware fixed in formaldehyde from the end of incubation, then embedded in paraffin blocs. Subsequently sections of 4 μm thickness were cut.

The melanin present in the skin sections was revealed by a specific staining called [symbol] Fontana Masson [symbol] . Said slides by condition were carried out with a magnification×400. The visualisable melanin on these slides was quantified by image analysis using the ImageJ program.

The results are expressed in form of the surface occupied by the melanin (number of pixels the grey scale of which is between 0 and 100; 0=black/255=white) in each slide (average +/− standard deviation, SD).

The statistical significance of the noted differences between the treated conditions and controls was assessed by a Student's test (*: p<0.05,**: p<0.01;***: p<0.001).

TABLE 7
Quantification of the melanic content of reconstructed melanized
human epidermal skins treated/untreated by image analysis:

| | | | ETOH BB | | | |
|---|---|---|---|---|---|---|
| WITH- | | | | | | 0.15 |
| OUT | Medium | α-MSH | | | | without |
| UV | alone | 100 nM | 0.05 | 0.15 | 0.3 | reapplication |
| Surface | 24131 | 38565 | 22754 | 29849 | 22821 | 22036 |
| occu- | 17119 | 25757 | 16628 | 15245 | 15368 | 19500 |
| pied | 30306 | 42233 | 16392 | 36248 | 20144 | 25949 |
| by the | 13966 | 55615 | 12954 | 7678 | 19722 | 16277 |
| melanin | 9673 | 51363 | 22308 | 6990 | 21260 | 11746 |
| (pixels) | 19100 | 43652 | 7362 | 10329 | 18916 | 19408 |
| | 13226 | 44188 | 4473 | 10212 | 20203 | 42503 |
| | 20874 | 44053 | 17132 | 22152 | 25844 | 19002 |
| | 26733 | 22738 | 17195 | 25753 | 17680 | 30479 |
| | 18715 | 34614 | 9717 | 15909 | 17895 | 14747 |
| Average | 19384.3 | 40277.8 | 14691.5 | 18036.5 | 19985.3* | 22164.7* |
| S.D. | 6370.9 | 10308.4 | 6034.9 | 10055.1 | 2911.3 | 8944.6 |
| % | 100 | 208 | 76 | 93 | 103 | 114 |
| medium | | | | | | |
| alone | | | | | | |
| % | — | — | 100 | 122.8 | 136 | 150.9 |
| ETOH | | | | | | |
| BB 0.05 | | | | | | |

### 3.—Results

The formulation “ETOH BB 0.15” increases significantly the melanin content of reconstructed melanized human epidermal skins, not exposed to UVA or to UVB, without re-application (+22% by biochemical analysis, +51% by image analysis).

The formulation “ETOH BB 0.3” increases significantly the melanin content in reconstructed melanized human epidermal skins, not exposed to UVA or to UVB (+28% by biochemical analysis, +36% by image analysis).

### EXAMPLE 7

### Effects on the Transport of Macromolecules

7.1—Assessment of the Rate of Transport of Macromolecules (Glucids, Lipids and Proteins) in a System of Isolated Microsomes. Direct Effect of the BB Product on Isolated Microsomes:

The microsomes of human keratinocytes form a membrane fraction obtained by high-speed differential centrifugation of a cell homogenate. This preparation of microsomes requires the addition of exogenous cofactors such as NADPH.

7.1.1—Transport of Glucids (Glucose)

The assay was performed in triplicate after the direct treatment of microsomes.
Charge 1: negative control not receiving any product.
Charge 2: positive control (0.25 mM phloretin,)
Charges 3-5: treated with the investigated product (3 concentrations)
Charges 6-8: treated with 0.25 mM phloretin and with the investigated product (3 concentrations)

The obtained microsomes from human keratinocytes in culture were washed 3 times with a PBS buffer without glucose then pre-incubated in 1 ml of the same buffer for 30 minutes at 37° C. Subsequently, this solution was discarded and the microsomes placed into the glucose-free PBS buffer containing 3-0-Methyl glucose (MG) and [³H] 3-0-MG with stirring in a water-bath at 37° C. The capture of 3-0-MG is stopped by addition of 1 ml of cold PBS containing cytochalasine B. The kinetics of incubation was performed between 30 and 120 seconds. The microsomes were further rinsed 2 times with PBS, then dissolved in NaOH (1M) at 4° C., over night. The radioactivity was determined with a scintillation counter.

The treatments of microsomes with the BB product alone or in the presence of phloretin were carried out at the same time as the introduction of [3H] 3-0-MG into the incubation medium.

The protein assay was performed following the BRADFORD method. The increase in absorbance at 595 nm, determined with spectrophotometer, is proportional to the protein concentration.

### Results:

Capture of [³H]3-O-MG
(nmol/mg proteins)

| | 30 s | 60 s | 90 s | 120 s |
|---|---|---|---|---|
| Negative control | 9 | 15 | 19 | 21 |
| BB 1/10 | 10 | 14 | 20 | 22 |
| BB 1/5 | 9 | 15 | 20 | 22 |
| BB 1/2 | 11 | 16 | 21 | 21 |
| Phloretin (0.25 mM) | 4 | 6 | 8 | 8 |
| BB | 5 | 11 | 17 | 19 |
| (1/10) + Phloretin | | | | |
| BB (1/5) + Phloretin | 6 | 12 | 18 | 21 |
| BB (1/2) + Phloretin | 7 | 14 | 18 | 22 |

The obtained results, after direct treatment of the microsomes of keratinocytes with the BB product in the used concentrations show no inhibition of the transport rate of glucose. The kinetics of glucose capture under physiological conditions is quasi-identical in the control microsomes and the microsomes treated with the BB product in the used 3 concentrations.

The direct treatment of microsomes with phloretin strongly inhibits the rate of transport of glucose. The treatment of microsomes with the BB product in the 3 concentrations simultaneously with phloretin significantly re-establishes the rate of transport of glucose. The kinetics of glucose capture, under inhibiting conditions, is perfectly re-established with the BB product in the 3 used concentrations.

7.1.2—Transport of Lipids

The assay was performed in triplicate after direct treatment of microsomes.
Charge 1: negative control not receiving any product.
Charge 2: positive control (1 mM Diphenhydramin)
Charge 3-5: treated receiving the investigated product (3 concentrations)
Charge 6-8: treated with 1 mM Diphenhydramin and with the investigated product (3 concentrations)

The obtained microsomes from human keratinocytes in culture were rinsed 3 times with 25 mM Tris buffer, then preincubated in 1 ml of the same buffer for 30 minutes at 37° C. This solution was then discarded and the microsomes were placed into the 25 mM Tris buffer containing [³H] Choline with stirring in a water bath at 37° C. The capture of choline was stopped by addition of a lysis buffer containing (50 mM de Tris, 140 mM NaCl, 1.5 mM MgSO₄, 0.5% Igepal-Ca-630, 0.2% SDS). The kinetics of incubation was performed between 30 and 120 seconds. Subsequently, the microsomes were rinsed 2 times with PBS then dissolved in NaOH (1M) at 4° C. over night. The radioactivity was determined with a scintillation counter.

The treatments of microsomes with the BB product alone or in the presence of Diphenhydramin (DPA) were carried out at the same time as the introduction of [³H] Choline into the incubation medium.

The protein assay was carried out following the BRADFORD method. The increase in absorbance at 595 nm, determined with a spectrophotometer, is proportional to the protein concentration.

### Results:

Capture of [³H]choline
(pmol/mg proteins)

| | 30 s | 60 s | 90 s | 120 s |
|---|---|---|---|---|
| Negative control | 155 | 201 | 261 | 301 |
| BB 1/10 | 175 | 212 | 268 | 310 |
| BB 1/5 | 163 | 210 | 277 | 321 |
| BB 1/2 | 194 | 240 | 289 | 312 |
| DPA (1 mM) | 74 | 105 | 132 | 142 |
| BB (1/10) + DPA | 100 | 121 | 153 | 183 |
| BB (1/5) + DPA | 113 | 131 | 168 | 199 |
| BB (1/2) + DPA | 125 | 140 | 189 | 223 |

The obtained results, after direct treatment of the microsomes of keratinocytes with the BB product in the used concentrations show no inhibition of the rate of transport of lipids. The kinetics of choline capture under physiological conditions is quasi-identical for the control microsomes and the microsomes treated with the BB product in the 3 used concentrations.

The obtained results show that the direct treatment of the microsomes with the Diphenhydramin strongly inhibits the rate of transport of lipids. The treatment of microsomes with the BB product in the 3 concentrations at the same time as with the Diphenhydramin restores the rate of transport of lipids. The kinetics of choline capture, under inhibiting conditions is moderately re-established with the BB product in the used 3 concentrations.

7.1.3—Transport of Proteins (Albumin)

The assay was performed in triplicate after the direct treatment of microsomes.
Charge 1: negative control not receiving any product
Charge 2: positive control (Nocodazole, 6 mg/ml)
Charge 3-5: treated receiving the investigated product (3 concentrations)
Charge 6-8: treated with Nocodazole 6 mg/ml, and with the investigated product (3 concentrations)

The obtained microsomes from human keratinocytes in culture were rinsed 3 times with a buffer, afterwards pre-incubated in 1 ml of the same buffer for 30 minutes at 3° C. This solution was subsequently discarded and the microsomes were placed into the HBSS buffer containing

albumin-FITC with stirring in a water bath at 37° C. The capture of albumin is stopped by addition of a Ringer's solution containing 122.5 mM NaCl; 5.4 mM KCl; 1.2 mM CaCl₂; 0.8 mM MgCl₂; 0.8 Na₂HPO₄; 0.2 NaHPO₄; 5.5 mM glucose; 10 mM HEPES, pH 7,4. Afterwards, the microsomes were treated with Triton X-100 at (0.1% v/v in 3-(N-morpholino) propanesulfonic acid 20 mM, pH 7.4). The fluorescence was quantified with a spectrophotometer (excitation 480 nm: emission 520 nm). The kinetics of incubation was performed between 30 and 120 seconds.

The treatment of microsomes with the BB product alone or in the presence of Nocodazole (ND) were carried out at the same time as the introduction of albumin-FITC into the incubation medium.

The assay of proteins was performed following the BRADFORD method. The increase in absorbance at 595 nm is proportional to the concentration of proteins measured with a spectrophotometer.

### Results:

Capture of albumin-FITC
(μg/mg proteins)

| | 30 s | 60 s | 90 s | 120 s |
|---|---|---|---|---|
| Negative control | 0.32 | 0.45 | 0.55 | 075 |
| BB 1/10 | 0.3 | 0.47 | 0.59 | 0.74 |
| BB 1/5 | 0.37 | 0.5 | 0.62 | 0.76 |
| BB 1/2 | 0.4 | 0.55 | 0.67 | 0.8 |
| ND (6 mg/ml) | 0.11 | 0.29 | 0.34 | 0.43 |
| BB (1/10) + ND | 0.12 | 0.3 | 0.33 | 0.41 |
| BB (1/5) + ND | 0.19 | 0.33 | 0.38 | 0.49 |
| BB (1/2) + ND | 0.23 | 0.38 | 0.44 | 0.57 |

The obtained results, after direct treatment of the microsomes of keratinocytes with the BB product in the used concentrations show no inhibition of the rate of transport of proteins. The kinetics of albumin capture under physiological conditions is quasi-identical for the control microsomes and the microsomes treated with the BB product in the 3 used concentrations.

The obtained results show that the direct treatment of microsomes with Nocodazole strongly inhibits the rate of transport of lipids. The treatment of microsomes with the BB product in concentrations ⅕ and ½ at the same time as the Nocodazole moderately re-establishes the rate of transport of lipids. No effect was observed in a concentration 1/10.

### 7.2—Assessment of the Rate of Transport of Macromolecules At the Level of Normal Human Keratinocytes

The applied method was that of explants allowing to obtain, from a biopsy of human skin, keratinocytes in primary cultures. The assays were carried out on keratinocytes between the 2nd and the 4th passage in order to ensure a reproductibility between the different experiences.

The keratinocytes were divided into multiwell plates (6 wells) at a rate of 10⁵ cells per well in 1 ml of culture medium SKINETHIC supplemented with insulin and hydrocortisone. The cells were incubated in the presence and absence of the investigated product.

7.2.1—Transport of Glucides (Glucose)

The assay was carried out in triplicate on normal human keratinocytes in culture.
Charge 1: negative control not receiving any product.
Charge 2: positive control (0.25 mM Phloretin)
Charge 3: treated receiving the investigated product
Charges 6-8: treated with 0.25 mM phloretin and the investigated product (3 concentrations)

The treatment of keratinocytes with the BB product in the presence and absence of 0.25 mM phloretin was carried out for 20 minutes at 37° C. The membrane microsomes were separated by differential centrifugation.

The same protocol of capture of [3H] 3-0-Methyl-Glucose was adopted for the determination of the rate of transport of glucose. (cf. 6.1.1).

### Results:

Capture of [³H]3-O-MG
(nmol/mg of proteins)

| | 30 s | 60 s | 90 s | 120 s |
|---|---|---|---|---|
| Negative control | 6 | 12 | 15 | 19 |
| BB 1/10 | 7 | 10 | 16 | 17 |
| BB 1/5 | 8 | 13 | 17 | 18 |
| BB 1/2 | 10 | 14 | 18 | 20 |
| Phloretin (0.25 mM) | 6 | 8 | 9 | 10 |
| BB | 6 | 10 | 15 | 18 |
| (1/10) + Phloretin | | | | |
| BB (1/5) + Phloretin | 7 | 13 | 16 | 19 |
| BB (1/2) + Phloretin | 8 | 13 | 17 | 18 |

The obtained results, after treatment of the normal keratinocytes with the BB product, prior to the separation of microsomes, show no inhibition of the rate of transport of glucose. The kinetics of capture of glucose under physiological conditions is quasi-identical for the control microsomes of keratinocytes and the microsomes of keratinocytes treated with the BB product in the used 3 concentrations.

The treatment of normal keratinocytes with phloretin, prior to the separation of microsomes, strongly inhibits the rate of transport of glucose. The treatment of normal keratinocytes with the BB product in the 3 concentrations at the same time as with phloretin re-establishes significantly the rate of transport of glucose. The kinetics of capture of glucose, under inhibiting conditions is perfectly restored with the BB product in the used 3 concentrations.

7.2.2—Transport of Lipids

The assay was carried out in triplicate on normal human keratinocytes in culture.
Charge 1: negative control not receiving any product.
Charge 2: positive control (1 mM Diphenhydramin)
Charge 3-5: treated receiving the investigated product (3 concentrations)
Charge 6-8: treated with 1 mM Diphenhydramin and with the investigated product (3 concentrations)

The treatment of keratinocytes with the BB product in the presence of 1 mM Diphenhydramin was carried out for 120 minutes at 37° C. The membrane microsomes were separated by differential centrifugation.

The same protocol of capture of [3H] choline was used for the determination of the speed of transport of choline (cf. 6.1.2)

### Results:

Capture of [³H]choline
(pmol/mg proteins)

| | 30 s | 60 s | 90 s | 120 s |
|---|---|---|---|---|
| Negative control | 142 | 173 | 202 | 215 |
| BB 1/10 | 151 | 163 | 198 | 223 |
| BB 1/5 | 162 | 178 | 207 | 227 |
| BB 1/2 | 174 | 191 | 218 | 231 |
| DPA (1 mM) | 92 | 115 | 143 | 156 |
| BB (1/10) + DPA | 112 | 128 | 159 | 174 |
| BB (1/5) + DPA | 119 | 132 | 165 | 179 |
| BB (1/2) + DPA | 127 | 139 | 174 | 191 |

The obtained results, after treatment of the normal keratinocytes with the BB product, prior to the separation of microsomes, show no inhibition of the rate of transport of lipids. The kinetics of capture of choline under physiological conditions is quasi-identical for the control microsomes of keratinocytes and the microsomes of keratinocytes treated with the BB product in the used 3 concentrations.

The treatment of normal keratinocytes with Diphenhydramin, prior to the separation of microsomes, moderately inhibits the rate of transport of lipids. The treatment of normal keratinocytes with the BB product in 3 concentrations at the same time as the Diphenhydramin restores the rate of transport of lipids. The kinetics of capture of choline, under inhibitory conditions, is moderately restored with the BB product in the used 3 concentrations.

7.2.3—Transport of Proteins (Albumin)

The assay was carried out in triplicate on normal human keratinocytes in culture.
Charge 1: negative control not receiving any product.
Charge 2: positive control (Nocodazole, 6 mg/ml)
Charge 3-5: treated receiving the investigated product (3 concentrations)
Charge 6-8: treated with Nocodazole, 6 mg/ml, and with the investigated product (3 concentrations)

The treatment of keratinocytes with the BB product in the presence and absence of Nocodazole, 6 mg/ml, was carried out for 120 minutes at 37° C. The membrane microsomes were separated by differential centrifugation.

The same protocol of capture of albumin-FITC was used for the determination of the rate of transport of albumin (cf. 3.1.4.2)

### Results:

Capture of albumin-FITC
(μg/mg proteins)

| | 30 s | 60 s | 90 s | 120 s |
|---|---|---|---|---|
| Negative control | 0.25 | 0.39 | 0.45 | 0.61 |
| BB 1/10 | 0.24 | 0.40 | 0.48 | 0.63 |
| BB 1/5 | 0.28 | 0.44 | 0.52 | 0.67 |
| BB 1/2 | 0.31 | 0.46 | 0.54 | 0.73 |
| S.D. (6 mg/ml) | 0.09 | 0.22 | 0.26 | 0.37 |
| BB (1/10) + S.D. | 0.1 | 0.24 | 0.26 | 0.39 |
| BB (1/5) + S.D. | 0.12 | 0.27 | 0.30 | 0.41 |
| BB (1/2) + S.D. | 0.16 | 0.29 | 0.36 | 0.47 |

The obtained results, after treatment of normal keratinocytes with the BB product, prior to the separation of microsomes, show no inhibition in the rate of transport of proteins. The kinetics of capture of albumin under physiological conditions is the same for control microsomes of keratinocytes and the microsomes of keratinocytes treated with the BB product in a concentration 1/10. A slight increase in the rate of transport of proteins is noted for concentrations ½ and ⅕.

The treatment of normal keratinocytes with Nocodazole, prior to the separation of microsomes, inhibits the rate of transport of proteins. The treatment of normal keratinocytes with the BB product in 2 concentrations at the same time as the Nocodazole re-establishes moderately the rate of transport of proteins. No effect was observed in the concentration 1/10.

### 7.3—Assessment of the Rate of Transport of Macromolecules At the Level of Human Keratinocytes in Senescence

The senescence is a phenomenon of cellular aging, where the cells are arrested in the phase G1 of the cell cycle and never enter the synthesis phase leading to the cell division. Senescent cells are characterized by their slow metabolism at synthesis and transport of macromolecules levels.

The cells in culture deprived of serum stop their growth, but continue to progress through the cell cycle until they reach the phase G1.

The applied method allowed to obtain, from a human skin biopsy, primary cultures of keratinocytes. The assays were performed on keratinocytes, between the 8th and the 10th passage, in order to ensure the presence of senescent cells at control cells level.

The keratinocytes were seeded in multiwell plates (6 wells) at a rate of 10⁵ cells per well in 3 ml of culture medium SKINETHIC supplemented with EGF, hydrocortisone, insulin and gentamicyn. Afterwards they are maintained for 5 days in incubator under CO₂.

7.3.1—Transport of Glucids (Glucose)

The assay was carried out in triplicate on human keratinocytes in senescence.
Charge 1: negative control not receiving any product.
Charge 2: positive control (0.25 mM Phloretin)
Charge 3: treated receiving the investigated product
Charges 6-8: treated with 0.25 mM phloretin and with the investigated product (3 concentrations)

The treatment of keratinocytes with the BB product in the presence and absence of 0.25 mM phloretin was performed for 20 minutes at 37° C. The membrane microsomes were separated by differential centrifugation.

The same protocol of capture of [3H] 3-0-Methyl-Glucose was applied to the determination of the rate of transport of glucose (cf. 6.1.1).

### Results:

Capture of [³H]3-O-MG
(nmol/mg of proteins)

| | 30 s | 60 s | 90 s | 120 s |
|---|---|---|---|---|
| Negative control | 3 | 5 | 10 | 11 |
| BB 1/10 | 4 | 8 | 12 | 12 |
| BB 1/5 | 6 | 8 | 13 | 13 |
| BB 1/2 | 7 | 10 | 14 | 15 |
| Phloretin (0.25 mM) | 1 | 3 | 5 | 7 |
| BB | 3 | 5 | 7 | 8 |
| (1/10) + Phloretin | | | | |
| BB (1/5) + Phloretin | 5 | 8 | 9 | 11 |
| BB (1/2) + Phloretin | 6 | 10 | 12 | 13 |

The obtained results show that the rate of transport of glucose is markedly low at the keratinocytes in senescence level compared to normal keratinocytes.

The treatment of keratinocytes in senescence with the BB product, prior to the separation of microsomes, shows an increase in the rate of transport of glucose compared to the untreated control cells. The kinetics of capture of glucose under physiological conditions is higher at the level of microsomes of keratinocytes treated with the BB product compared to control microsomes of keratinocytes. This result is obtained for different incubation periods (30, 60, 90 and 120 seconds).

The treatment of keratinocytes in senescence with phloretin, prior to the separation of microsomes, strongly inhibits the rate of transport of glucose. The treatment of normal keratinocytes with the BB product in 2 concentrations (⅕ and ½) at the same time as with phloretin restores significantly the rate of transport of glucose. A slight effect was observed in the concentration 1/10.

7.3.2—Transport of Lipids

The assay was carried out in triplicate on human keratinocytes in senescence.
Charge 1: negative control not receiving any product.
Charge 2: positive control (1 mM Diphenhydramin)
Charge 3-5: treated receiving the investigated product (3 concentrations)
Charge 6-8: treated with 1 mM Diphenhydramin and with the investigated product (3 concentrations)

The treatment of keratinocytes with the BB product in the presence and absence of 1 mM Diphenhydramin was carried out for 120 minutes at 37° C. The membrane microsomes were separated by differential centrifugation.

The same protocol of capture of [3H] Choline was applied to the determination of the rate of transport of choline (cf. 6.1.2)

### Results:

Capture of [³H]choline
(pmol/mg of proteins)

| | 30 s | 60 s | 90 s | 120 s |
|---|---|---|---|---|
| Negative control | 114 | 132 | 164 | 181 |
| BB 1/10 | 110 | 135 | 166 | 173 |
| BB 1/5 | 113 | 143 | 188 | 194 |
| BB 1/2 | 121 | 153 | 187 | 201 |
| DPA (1 mM) | 55 | 79 | 106 | 119 |
| BB (1/10) + DPA | 52 | 69 | 110 | 125 |
| BB (1/5) + DPA | 67 | 87 | 132 | 141 |
| BB (1/2) + DPA | 77 | 104 | 137 | 148 |

The obtained results show that the rate of transport of lipids is markedly lower at keratinocytes in senescence level compared to normal keratinocytes.

The treatment of keratinocytes in senescence with the BB product, prior to the separation of microsomes, shows no inhibition of the rate of transport of lipids compared to untreated control cells. The kinetics of capture of lipids under physiological conditions is comparable for the microsomes of keratinocytes treated with the BB product and the control microsomes of keratinocytes.

The treatment of keratinocytes in senescence with Diphenhydramin, prior to the separation of microsomes, strongly inhibits the rate of transport of lipids. The treatment of keratinocytes in senescence with the BB product in 2 concentrations (⅕ and ½) at the same time as with Diphenhydramin re-establishes significantly the rate of transport of lipids. No effect was observed in the concentration 1/10.

7.3.3—Transport of Proteins (Albumin)

The assay was carried out in triplicate on human keratinocytes in senescence.
Charge 1: negative control not receiving any product.
Charge 2: positive control (Nocodazole, 6 mg/ml)
Charge 3-5: treated receiving the investigated product (3 concentrations)
Charge 6-8: treated with Nocodazole, 6 mg/ml, and the investigated product (3 concentrations)

The treatment of keratinocytes with the BB product in the presence and absence of Nocodazole, 6 mg/ml, was carried out for 120 minutes at 37° C. The membrane microsomes were separated by differential centrifugation.

The same protocol of capture of albumin-FITC was applied to determine the rate of transport of albumin (cf. 6.1.3)

### Results:

Capture of albumin-FITC
(μg/mg of proteins)

| | 30 s | 60 s | 90 s | 120 s |
|---|---|---|---|---|
| Negative | 0.17 | 0.29 | 0.35 | 0.49 |
| control | | | | |
| BB 1/10 | 0.18 | 0.34 | 0.39 | 0.52 |
| BB 1/5 | 0.18 | 0.36 | 0.41 | 0.54 |
| BB 1/2 | 0.2 | 0.37 | 0.43 | 0.55 |
| ND (6 mg/ml) | 0.06 | 0.11 | 0.19 | 0.3 |
| BB (1/10) + ND | 0.05 | 0.12 | 0.22 | 0.33 |
| BB (1/5) + ND | 0.09 | 0.14 | 0.27 | 0.38 |
| BB (1/2) + ND | 0.1 | 0.19 | 0.3 | 0.43 |

The obtained results demonstrate that the rate of transport of proteins is markedly lower at the keratinocytes in senescence level compared to normal keratinocytes. The treatment of keratinocytes in senescence with the BB product, prior to the separation of microsomes, shows no inhibition of the rate of transport of proteins compared to the untreated control cells. The kinetics of capture of albumin under physiological conditions is comparable for the microsomes of keratinocytes treated with the BB product and the control microsomes of keratinocytes.

The treatment of keratinocytes in senescence with Nocodazole, prior to the separation of microsomes, inhibits the rate of transport of proteins. The treatment of keratinocytes in senescence with the BB product in 2 concentrations (⅕ and ½), at the same time as with Nocodazole, restores significantly the rate of transport of proteins. A slight effect is observed with the concentration 1/10.

### EXAMPLE 8

### Effect on the Respiration and the Synthesis of ATP

### 8.1—Study of the Effect of the Product on Mitochondrial And Cellular Respiration

The amount of dissolved oxygen in a solution can be determined with a Clark electrode. The oxygen diffusing through a teflon film will be reduced at the level of the platinum cathode polarized at −0.8 Volt. Under these conditions, the current passing between this cathode and the silver anode is proportional to the concentration of oxygen in the solution. The ionic bridge between both electrodes is created by a saturated solution of KCl.

The acquisition and the treatment of measurements are carried out with a microcomputer (IBM-PC) in real time. A program allows to continuously visualize the oxygen amount in the tank and the instantaneous derivative as well corresponding to the rate of oxygen consumption calculated in real time.

Five concentrations were tested and the determinations were carried out in triplicate. This study is carried out depending on two different conditions:

8.1.1—Effect of the Product on Cellular Respiration: Measurement of Basal Respiration

Effect on the rate of basal respiration on non permeabilized cells in the presence of glucose.

The keratinocytes were cultured in incubator with CO₂, at a rate of 10⁶ per run in a culture medium DMEM supplemented with hydrocortisone, EGF and FCS (10%).

This protocol was carried out by direct application of the product on the cells in the tank of oxygraph.

The cells (keratinocytes), in a concentration of 10⁶ cells/ml, are suspended in a “respiration buffer” (Hanks-Hepes 20 mM glucose), in the tank of oxygraph thermostatised at 30° C. and equipped with a Clark electrode (1 ml of respiration buffer containing under these conditions 480 oxygen atoms).

Under these conditions, a rate of oxygen consumption (basal respiration of cells) can be measured. The addition of different amounts of the product (final concentrations: 1/10; ⅕ and ½) in the tank of oxygraph allows to reveal a possible stimulation or inhibition of this breathing.

All determinations are resumed in the following Table hereafter.

| | Tested product (BB) | | | |
|---|---|---|---|---|
| | Control | 1/10 | 1/5 | 1/2 |
| Rate of | 3.64 ± 0.37 | 3.72 ± 0.31 | 4.26 ± 0.25 | 4.75 ± 0.50 |
| respiration | | | | |
| (n atom O/min/ | | | | |
| 10⁶ cells) | | | | |
| n = 3 | | | | |
| % of basal | 100 ± 6 | 102 ± 4 | 117 ± 3 | 130 ± 5 |
| respiration | | | | |
| n = 3 | | | | |

Under the applied experimental conditions and considering the obtained results, the product in contact with the keratinocytes induces a significant increase in the rate of basal respiration in concentrations (⅕ and ½).

8.1.2.—Effect of the Product on Mitochondrial Respiration:

Effect on the rate of respiration of permeabilized cells in the presence of the respiration substrate, pyruvate-malate, in order to assess the mitochondrial respiration.

The keratinocytes were cultured in incubator under CO₂, at a rate of 10⁶ cells per run, in a culture medium DMEM supplemented with hydrocortisone, EGF and FCS (10%).

This protocol was carried out by direct application of the product onto the cells in the tank of oxygraph.

The cells (keratinocytes), in a concentration of 10⁶ cells/ml, are suspended in a “respiratory buffer” (Hanks-Hepes 20 mM glucose), in the tank of oxygraph thermostatized at 30° C. and equipped with a Clark electrode (1 ml respiratory buffer containing under these conditions 480 oxygen atoms). The cells are permeabilized with digitonin. The addition of a respiratory substrate (10 mM pyruvate and 10 mM malate) allows to observe a rate of oxygen consumption (stage 2 according to Chance). The addition of different amounts of the product (final concentrations: 1/10; ⅕ and ½) into the tank of oxygraph allows to reveal a possible stimulation or inhibition of this respiration.

All determinations are resumed in the Table hereafter.

| | Tested product (BB) | | | |
|---|---|---|---|---|
| | Control | 1/10 | 1/5 | 1/2 |
| Rate of | 2.67 ± 0.21 | 2.83 ± 0.15 | 3.25 ± 0.11 | 3.52 ± 0.09 |
| respiration | | | | |
| (n atom O/min/ | | | | |
| 10⁶ cells) | | | | |
| n = 3 | | | | |
| % of basal | 100 ± 2 | 106 ± 4 | 122 ± 3 | 132 ± 5 |
| respiration | | | | |
| n = 3 | | | | |

Under the applied experimental conditions and considering the obtained results, the product in contact with human keratinocytes in culture induces a significant increase in the rate of mitochondrial respiration in concentrations (⅕ and ½).

### 8.2—Study of the Effect of the Product on Mitochondrial ATP Synthesis

The measurement is carried out with a device of Luminoscan type using the ATP monitoring reagent (ATP Bioluminescence Assay Kit HS II) by Boehringer Mannheim. The amount of ATP present in this aliquot can be determined thanks to the following enzymatic reaction:

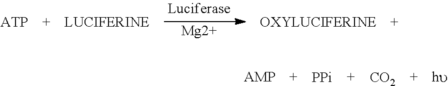

The intensity of the emitted light during this reaction can be measured with a luminometer (Luminoscan) which transcribes it in RLU (relative luminosity units). The measured RLU can be converted into mols of ATP with reference to a range of standards of ATP.

The rate of synthesis of ATP is expressed in mmols/min/10⁶ cells.

Five concentrations were tested and the determinations were carried out in triplicate.

The keratinocytes were cultured in incubator under CO₂, at a rate of 10⁶ per run in a culture medium DMEM supplemented with hydrocortisone, EGF and FCS (10%).

This protocol was carried out by direct application of the product onto the cells in the oxygraph tank.

The cells (keratinocytes), in a concentration of 10⁶ cells/ml, are suspended in a “respiration buffer” (Hanks-Hepes 20 mM glucose), in the tank of oxygraph thermostatized at 30° C. The cells are impermeabilized with digitonin. The addition of a respiratory substrate (10 mM pyruvate and 10 mM malate) allows to observe a rate of oxygen consumption (stage 2 according to Chance). After addition of different amounts of the product (concentrations: 1/10; ⅕ and ½) into the tank of oxygraph and at regular intervals, an aliquot is taken in the tank of oxygraph for the ATP assay following the method described hereabove. The addition of different amounts of the product into the tank of oxygraph, allows to reveal a possible activation or inhibition of the ATP synthesis. All determinations are resumed in the following Table herebelow.

| | Tested product (BB) | | | |
|---|---|---|---|---|
| | Control | 1/10 | 1/5 | 1/2 |
| Rate of ATP | 5.78 ± 0.25 | 6.16 ± 0.34 | 6.96 ± 0.29 | 7.24 ± 0.54 |
| synthesis | | | | |
| (nmol/min/ | | | | |
| 10⁶ cells) | | | | |
| n = 3 | | | | |
| % of ATP | 100 ± 3 | 107 ± 5 | 120 ± 8 | 125 ± 6 |
| synthesis n = 3 | | | | |

Under the applied experimental conditions and considering the obtained results, the product in contact with human keratinocytes in culture induces a significant increase in the rate of mitochondrial ATP synthesis in concentrations (⅕ and ½).

### 8.3—Study of the Effect of the Product on Energetic Metabolism

Assessment of the effect of the product at the energetic metabolism level by measurement of the concentrations of adenylic nucleotides. The study was carried out on primary cultures of human keratinocytes treated for 5 days prior to the study.

The amount of ATP, ADP and AMP contained in the cells is measured by High Pressure Liquid Chromatography (HPLC).

On a Beckman device, a column (10×0.46 cm) with a support Spherisorb NH₂ with a granulometry of 5 μm was used. The elution solvent is a potassium phosphate solution; depending on the molarity and the pH thereof, the retention time of nucleotides is more or less long. The rate of elution is of 1 ml/min. The elution profile is monitored by measuring the absorbance at 254 nm in isocratic.

A range of standards is carried out between 0.1 and 1 nmol of ATP, of ADP and of AMP measuring the surface of the area under the respective maxima.

Five concentrations were tested and the determinations were carried out in triplicate.

The ATP, ADP and AMP concentrations of a cellular extract (10⁶ cells/ml) are assayed by HPLC. They are expressed in nmol/min/mg proteins. The energetic charge (E. C.) is calculated following the formula:

([ATP]+½[ADP])/([ATP]+[ADP]+[AMP])

The calculation of the energetic charge is carried out in triplicate on control charges and on treated charges.

All determinations are resumed in the Table hereafter.

The ATP, ADP and AMP concentrations are expressed in nmol/mg proteins (n=3).

| | Tested BB product | | | |
|---|---|---|---|---|
| | Control | 1/10 | 1/5 | 1/2 |
| [ATP] | 4214 | 4060 | 4554 | 4860 |
| [ADP] | 936 | 1103 | 1162 | 936 |
| [AMP] | 673 | 488 | 719 | 1178 |
| [ATP/ADP] | 4.50 | 3.68 | 3.92 | 5.19 |
| Total | 5823 | 5651 | 6434 | 6973 |
| E.C. | 0.804 | 0.816 | 0.798 | 0.764 |

Under the applied experimental conditions and considering the obtained results, the product in contact with human keratinocytes in culture induces no modification of the energetic load in the used concentrations of the product.

1. A process for obtaining freeze-dried product of gametophyte cells of brown algae comprising the steps of:
harvesting mature sporophytes of brown algae;
emitting spores from said sporophytes in vitro;
germinating said spores in vitro;
harvesting gametophyte cells; and
freeze-drying the harvested gametophyte cells to obtain a freeze-dried product of gametophyte cells of brown algae.

2. The process according to claim 1, wherein the germinating is carried out for 6 to 20 days.

3. The process according to claim 1, wherein the harvesting gametophyte cells is carried out 10 to 12 days after germinating.

4. The process according to claim 1, further comprising inhibiting germination of the harvested gametophyte cells prior to freeze-drying.

5. A cosmetic preparation for topical use which contains as an active ingredient a freeze-dried product of gametophytes of brown algae obtained by the process comprising the steps of:
harvesting mature sporophytes of brown algae;
emitting spores from said sporophytes in vitro;
germinating said spores in vitro;
harvesting gametophyte cells; and
freeze-drying the harvested gametophyte cells to obtain a freeze-dried product of gametophyte cells of brown algae.

6. The cosmetic preparation according to claim 5, wherein the freeze dried product is 0.2 to 5% by weight of the preparation.

7. The cosmetic preparation according to claim 5,
wherein the gametophyte cells are obtained from the species selected from the group consisting of Laminaria saccharina, Laminaria hyperborea, Alaria esculenta, and Undaria pinnatifida.
